(19) United States
(12) Patent Application Publication
(10) Pub. No.: US 2015/0150932 A1
(43) Pub. Date: Jun. 4, 2015

(54) SOLUABLE FIBER DIGESTIVE THERAPY AND DELIVERY SYSTEM

(71) Applicant: William H. Miller, Missoula, MT (US)

(72) Inventor: William H. Miller, Missoula, MT (US)

(73) Assignee: W.F. YOUNG, INCORPORATED, EAST LONGMEADOW, MA (US)

(21) Appl. No.: 14/554,220

(22) Filed: Nov. 26, 2014

Related U.S. Application Data
(60) Provisional application No. 61/911,255, filed on Dec. 3, 2013.

(51) Int. Cl. A61K 36/899; A61K 36/68; A23L 1/308; A61K 36/42

(52) U.S. Cl. CPC A61K 36/899; A61K 36/42; A61K 36/68; A23L 1/3081; A23V 2002/00

(57) ABSTRACT

A delivery system in the form of a soluble fiber formulation for therapeutic supplement and medication delivery includes approximately 70%-80% water, 14%-48% pumpkin powder, 0.5%-2.5% oat fiber powder and 0.4%-1.0% psyllium powder. The formulation also includes 0.6%-0.7% sodium bisulfate 0.0%-0.5% flavoring and 0.1%-0.25% potassium sorbate. The ingredients are mixed together to form a puree consistency.

### CROSS-REFERENCE TO RELATED APPLICATIONS

This application claims the benefit of U.S. Provisional Application Serial No. 61/911,255, filed on Dec. 3, 2013, which is herein incorporated by reference in its entirety.

### FIELD OF THE INVENTION

The present invention relates generally to a delivery system and, more particularly, to a digestive therapy and delivery system in the form of a soluble fiber formulation for therapeutic supplement and medication delivery.

### BACKGROUND OF THE INVENTION

Humans increasingly rely on dietary supplements, nutritional supplements and medications to treat a wide variety of conditions and specific health issues and/or to improve general health and well-being. For example, fiber supplements are a popular means of treating digestive issues or recurring digestive problems. Often times, supplements and medications for treating or managing such issues are administered orally in the form of a pill, tablet or capsule.

Notably, the absorption rate of pills and capsules is only 5% to 18%, which means that approximately 80-95% of the ingredients in pills are excreted from the body, which in itself can be very difficult on the body. Translated into monetary terms, for every $100 spent on pills, approximately $90 may end up being voided by the body. In addition, medications or supplements administered in pill or capsule form may cause irritation and discomfort for those afflicted with irritable bowel syndrome, hiatal hernias, diverticulitis and other digestive system issues.

As will be readily appreciated, household pets such as canines, cats, ferrets and other companion animals are not immune to digestive or other health conditions. Indeed, humans have become increasingly aware of the health and specific medical issues of their pets and often turn to dietary supplements, nutritional supplements and medications to treat such conditions. As with humans, such supplements and medications are often administered to pets in pill form.

The anatomy of canines and other household pets and domestic animals is different from that of humans, which requires special considerations in the manner and means by which supplements and medications are orally administered to treat such conditions. With canines, specifically, digestion begins in the mouth, where saliva lubricates the food and passes it down the esophagus. A canine's teeth, including molars, are sharp and jagged, and are designed for gripping and tearing, but are not well-suited for grinding. In addition, a canine's jaws are incapable of moving sideways, thereby limiting a canine's ability to grind food. Accordingly, the ability of a canine to chew and grind supplements or medications administered in pill form, which is often necessary for efficacy, is substantially limited.

Moreover, canines have a gulping reflex and very short digestive tracks. This results in supplements and medications in pill form not being able to be adequately digested and absorbed in the bloodstream before they are voided, reducing efficacy.

In view of the above, there is a need for a supplement and medication delivery system for canines and other animals that is more effective and has a higher rate of absorption than supplements and medications administered in pill or capsule form.

### SUMMARY OF THE INVENTION

It is an object of the present invention to provide a delivery system for therapeutic supplements and medications.

It is an object of the present invention to provide a delivery system for therapeutic supplements and medications in the form of a soluble fiber formulation.

It is another object of the present invention to provide a soluble fiber formulation that promotes overall health and longevity in canines and other companion animals.

It is another object of the present invention to provide a soluble fiber formulation that helps relieve both constipation and diarrhea in canines and other companion animals.

It is another object of the present invention to provide a soluble fiber formulation for therapeutic supplements and medication delivery that has a high level of digestibility.

It is another object of the present invention to provide a soluble fiber formulation for therapeutic supplement and medication delivery that has a higher rate of absorption than supplements and medications administered in pill form.

It is another object of the present invention to provide a soluble fiber formulation that promotes a soft, moist stool.

It is another object of the present invention to provide a soluble fiber formulation for therapeutic supplement and medication delivery that is easy to administer.

According to an embodiment of the present invention, a digestive therapy and delivery composition is provided. The composition includes pumpkin powder within a range of from about 14.0 to about 18.0 percent by weight, oat fiber powder within a range of from about 0.5 to about 2.5 percent by weight, psyllium powder within a range of from about 0.4 to about 1.0 percent by weight, and moisture within a range of from about 70.0 to about 80.0 percent by weight.

According to another embodiment of the present invention, a method for manufacturing a digestive therapy and delivery composition is provided. The method includes the steps of providing a base material with 14.0 to 18.0 percent by weight of pumpkin powder, 0.5 to 2.5 percent by weight of oat fiber powder, and 0.4 to 1.0 percent by weight of psyllium powder, mixing the base material with about 70.0 to about 80.0 percent by weight of water to form a puree, and heating the puree to greater than 180 degrees Fahrenheit.

According to yet another embodiment of the present invention a method of manufacturing a composition for digestive therapy and therapeutic supplement and medication delivery in a companion animal is provided. The method includes the steps of providing 70.0 to 80.0 percent by weight of liquid, and mixing 14.0 to 18.0 percent by weight of pumpkin powder, 0.5 to 2.5 percent by weight of oat fiber powder, and 0.4 to 1.0 percent by weight of psyllium powder with the liquid to form a puree.

### BRIEF DESCRIPTION OF THE DRAWINGS

The present invention will be better understood from reading the following description of non-limiting embodiments, with reference to the attached drawings, wherein below:

FIG. 1 is a flow diagram illustrating a method for manufacturing a digestive therapy and delivery composition in accordance with an embodiment of the present invention.

### DETAILED DESCRIPTION OF THE PREFERRED EMBODIMENT

In accordance with the present invention, a soluble fiber delivery system is provided in which dietary supplements, nutritional supplements, therapeutic supplements and medications can be administered to animals with increased ease and efficacy. Preferably, the delivery system includes a soluble fiber formulation comprised principally of a blend of highly soluble fibers and insoluble fiber. The formulation also includes preservatives to prevent decomposition by microbial growth or by undesirable chemical changes.

The following example demonstrates the present invention. All ingredient percentages refer to a measurement by weight.

### Example 1

### Base Formulation for Supplement and Medication Delivery

| Ingredient | Percentage |
|---|---|
| Water | 70%-80% |
| Pumpkin Powder | 14%-18% |
| Oat Fiber Powder | 0.5%-2.5% |
| Psyllium Powder | 0.4%-1.0% |
| Sodium Bisulfate | 0.6%-0.7% |
| Potassium Sorbate | 0.1%-0.25% |
| Flavoring | 0.0%-0.5% |

Ingredients are mixed with water to form a puree consistency. In an embodiment, the ingredients are mixed to form a puree consistency having a viscosity about the same as toothpaste. The puree is then heated to a temperature of greater than approximately 180 degrees Fahrenheit, and more preferably to approximately 190 degrees Fahrenheit and hot packed into a delivery mechanism. In an embodiment, the delivery mechanism is a 113 g (4 oz.) foil gusset bottom pouch with a spout and screw cap.

Importantly, the above-described base formulation serves as an effective digestive therapy for animals such as canines and other companion animals. In particular, the pumpkin powder, oat fiber powder and psyllium powder form a blend of highly soluble fibers and insoluble fiber that have been shown to prevent cancer, improve blood sugar, aid digestion, promote weight loss and support growth of friendly, healthy gastrointestinal micro flora. In this respect, the formulation of the present invention promotes overall health and longevity, especially in canines.

As will be readily appreciated, soluble fiber helps digestion and firms up loose stools by ‘holding’ water. This thickening characteristic has also been shown to soothe the stomach. In addition, soluble fiber also aids in bowel functions, meaning that it soothes the digestive tract and helps calm down any contractions that may take place within the intestines and, therefore, helps relieve both constipation and diarrhea. The formulation of the present invention, therefore, improves the digestive system and brings balance to an animal's bowels.

In particular, the formulation of the present invention functions as a stool softener which relieves constipation, and aids in alleviating upset stomach or indigestion in the animal. In addition, the soluble fiber in the formulation helps absorb the excess water in the bowels, thereby preventing diarrhea, while also hydrating to prevent dehydration. More specifically, when soluble fiber passes through the colon, it absorbs excess liquid, thus preventing a case of diarrhea by creating a gel-like substance before it is excreted. As a result, when the muscles around the colon have to stretch, it gives something to hold on to during contractions, giving the rectum a more relaxed release.

This relaxed release is important in canines, as it keeps the anal glands working well. In particular, periodic and healthy expression of the anal glands requires that the canine's stool be a particular consistency. If the stool is too hard, the anal glands can become irritated, and if the stool is too soft, the anal glands may not be expressed, leading to discomfort. The formulation of the present invention, therefore, aids in maintaining a canine's stool at an optimum consistency for healthy anal gland expression.

In addition to the above-described advantages in alleviating constipation and diarrhea, the formulation of the present invention increases the speed with which food waste products are eliminated from the body, thus reducing the body's exposure to free radicals and other toxins. The soluble fiber in the formulation acts like a sponge and binds to bile salts and other waste products carried out in the stool, thus preventing their absorption into the blood stream.

At the same time, however, soluble fiber also keeps food in the stomach longer, which slows down the absorption of carbohydrates after eating, leading to the stabilization of blood sugar. The formulation may also facilitate weight loss when administered to overweight animals, as the soluble fiber contained therein gives the animal a feeling of being full, yet contains no calories. In particular, the soluble fiber absorbs and holds a significant amount of water which reduces the amount the animal eats at each meal.

Importantly, the pumpkin powder contained in the formulation contains antioxidant vitamins which are important for overall health. In particular, the formulation contains Vitamin A, which is essential for immune system health, skin and coat, as well as the lining of the digestive and respiratory tracts. The formulation, by the inclusion of pumpkin powder, is also high in Vitamin C, Calcium and Iron.

In addition to the above-described advantages and myriad benefits in terms of promoting overall health and digestive system balance, the formulation of the present invention has been found to be extremely palatable. That is, the formulation is readily ingested by subject animals without resistance, thereby providing an ease of application heretofore unknown in the art.

In an embodiment, the above-described base formulation may be utilized as a delivery system for various supplements in addition to serving as an effective digestive therapy. In particular, it is contemplated that the base formulation of the present invention may be mixed with, and serve as a delivery system for, one or more of Glucosamine, Chondroitin, Flax, L-Theanine. As will be readily appreciated, the base formulation is not limited to serving as a delivery system for only these supplements and medications. Indeed, it is contemplated that the base formulation described above may be utilized as a delivery system for many other nutritional, dietary and/or therapeutic supplements or medications. Importantly, it has been discovered that various supplements or medications can be added to the base formulation without negating or diminishing the beneficial effects of the formulation in terms of absorption rate, stool consistency optimization and the like, and without altering the efficacy and desired benefits of the supplements or medications. In fact, as discussed hereinafter, efficacy of the supplements or medications added to the formulation has been found to increase relative to the efficacy of supplements and medications administered in pill form. In certain cases where the supplements or medications to be delivered are not heat tolerant, the base formulation will be mixed at ambient temperatures and then irradiated.

In certain embodiments, the flavoring may include natural and/or artificial flavorings. In an embodiment, the base formulation may be augmented with other natural and/or artificial flavors to provide additional and more complex flavor profiles.

The formulation of the present invention, therefore, serves dual functions; as an effective digestive therapy, as well as a delivery system for supplements and medications.

In addition to these advantages, the formulation for supplement and medication delivery of the present invention has numerous advantages over dosing with pills, especially when administered to canines. For example, the absorption rate of the formulation of the present invention, and the supplements or medications delivered thereby, is up to about 98%. Accordingly, fewer doses are therefore required for efficacy. In addition, the formulation of the present invention, in puree form, is much simpler to swallow, particularly for canines.

In contrast to pills, the ingredients in the formulation of the present invention are not compressed, which requires additional digestion before assimilation into the blood stream. In connection with this, the puree formulation of the present invention optimizes the absorption of supplements and medication, as it is quickly absorbed into the blood stream, resulting in faster (desired) effects. Moreover, unlike pills, purees are not powdered. This means that liquids do not require any sort of buffers, binders or fillers, which would cause additional delay in digestions; in other words, taking more time to enter the blood stream. Lastly, the pure formulation of the present invention does not ‘clog’ sewers, cesspools or septic systems, as up to 98% of the liquids are absorbed. This is in contrast to pills wherein only 10% of pills are absorbed, leaving roughly 90% of pills ending up in waste systems.

As will be readily appreciated, therefore, the present invention provides a delivery system that includes a soluble fiber formulation in the form of a puree that allows for dietary supplements, nutrients, and medications to be more readily absorbed into the digestive system and which enhances the speed of absorption through the digestive system. As discussed above, additional ingredients may be added to the formulation to create a multitude of supplement or medicinal products that can be quickly, easily and effectively administered for veterinary use, including to livestock, equine, domestic and companion animals.

While the soluble fiber digestive therapy and delivery system described above has been particularly described as being manufactured and administered as a puree, it is contemplated that it can take other forms including, but not limited to, powders, liquids and gels. In particular, it is contemplated that the digestive therapy and delivery system may be in any form suitable as a means of introducing soluble fiber into an animal's digestive system.

While the soluble fiber digestive therapy and delivery system of present invention is contemplated for use particularly with companion animals such as dogs, cats, ferrets and other companion and domestic animals, as discussed above, the present invention is not so limited in this regard. In particular, it is contemplated that the present invention may be utilized as an effective digestive therapy and/or a delivery system for supplements and medications for humans. In connection with this, it is foreseen and contemplated that the formulation of the present invention, when administered to humans, can provide substantially similar benefits in terms of supplement and medication delivery and the promotion of digestive system health and balance.

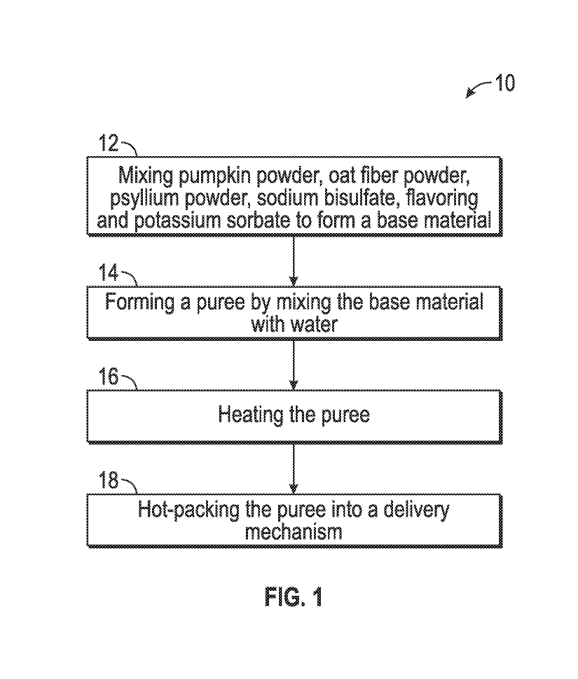

As discussed above, and with further reference to FIG. 1, a method 10 for manufacturing a digestive therapy and delivery composition is illustrated. As shown therein, at step 12, a base material is constructed by mixing 14.0 to 18.0 percent by weight of pumpkin powder, 0.5 to 2.5 percent by weight of oat fiber powder, 0.4 to 1.0 percent by weight of psyllium powder, 0.6 to 0.7 percent by weight of sodium bisulfate, 0.0 to 0.5 percent by weight of flavoring, and 0.1 to 0.25 percent by weight of potassium sorbate. The base material is then mixed with about 70.0 to about 80.0 percent by weight of water to form a puree, at step 14. As step 16, the puree is then heated. Finally, at step 18, the puree is hot-packed the into a delivery mechanism, such as a pouch.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of this disclosure.

What is claimed is:

1. A digestive therapy and delivery composition, comprising:
pumpkin powder within a range of from about 14.0 to about 18.0 percent by weight;
oat fiber powder within a range of from about 0.5 to about 2.5 percent by weight;
psyllium powder within a range of from about 0.4 to about 1.0 percent by weight; and
moisture within a range of from about 70.0 to about 80.0 percent by weight.

2. The composition of claim 1, further comprising:
sodium bisulfate within a range of from about 0.6 to 0.7 percent by weight.

3. The composition of claim 2, further comprising:
flavoring within a range of from about 0.0 to about 0.5 percent by weight.

4. The composition of claim 3, further comprising:
potassium sorbate within a range of from about 0.1 to about 0.25 percent by weight.

5. The composition of claim 1, wherein:
the flavoring includes at least one of a natural flavoring and an artificial flavoring.

6. A method for manufacturing a digestive therapy and delivery composition, comprising the steps of:
providing a base material with 14.0 to 18.0 percent by weight of pumpkin powder, 0.5 to 2.5 percent by weight of oat fiber powder, and 0.4 to 1.0 percent by weight of psyllium powder;
mixing the base material with about 70.0 to about 80.0 percent by weight of water to form a puree; and
heating the puree to greater than approximately 180 degrees Fahrenheit.

7. The method according to claim 6, further comprising the step of:
hot-packing the composition into a delivery mechanism.

8. The method according to claim 7, wherein:
the delivery mechanism is a foil gusset bottom pouch having a spout and a cap.

9. The method according to claim 6, wherein:
the base material further includes 0.6 to 0.7 percent by weight of sodium bisulfate, 0.0 to 0.5 percent by weight of flavoring, and 0.1 to 0.25 percent by weight of potassium sorbate.

10. The method according to claim 6, further comprising the step of:
adding at least one of a natural flavoring and an artificial flavoring to the base material.

11. The method according to claim 6, further comprising the step of:
mixing at least one of glucosamine, chondroitin, flax and L-Theanine into the composition.

12. The method according to claim 6, further comprising the step of:
mixing at least one nutritional supplement or medication into the composition.

13. A method of manufacturing a composition for digestive therapy and therapeutic supplement and medication delivery in a companion animal, comprising the steps of:
providing 70.0 to 80.0 percent by weight of liquid; and
mixing 14.0 to 18.0 percent by weight of pumpkin powder, 0.5 to 2.5 percent by weight of oat fiber powder, and 0.4 to 1.0 percent by weight of psyllium powder with the liquid to form a puree.

14. The method according to claim 13, further comprising the step of:
heating the puree to greater than about 180 degrees Fahrenheit.

15. The method according to claim 13, wherein:
the liquid is water.

16. The method according to claim 13, further comprising the step of:
mixing 0.6 to 0.7 percent by weight of sodium bisulfate, 0.0 to 0.5 percent by weight of flavoring, and 0.1 to 0.25 percent by weight of potassium sorbate with the liquid.

17. The method according to claim 16, further comprising the step of:
hot-packing the puree into a pouch for delivery.

18. The method according to claim 16, further comprising the step of:
mixing at least one nutritional supplement or medication into the puree.
